(19) United States
(12) Patent Application Publication
(10) Pub. No.: US 2016/0279280 A1
(43) Pub. Date: Sep. 29, 2016

(54) Macroporous Granules of Alkaline Earth Phosphates Using Cement Technology and Gas Evolving Porogen

(71) Applicant: THE UNIVERSITY OF TOLEDO, Toledo, OH (US)

(72) Inventors: Sarit B. BHADURI, Toledo, OH (US); Huan ZHOU, Toledo, OH (US); Anand K. AGARWAL, Toledo, OH (US); Vijay K. GOEL, Toledo, OH (US); Sameh SALEH, Toledo, OH (US)

(73) Assignee: The University of Toledo, Toledo, OH (US)

(21) Appl. No.: 15/038,218

(22) PCT Filed: Nov. 21, 2014
(86) PCT No.: PCT/US2014/066761

§ 371 Date: May 20, 2016

Related U.S. Application Data
(60) Provisional application No. 61/907,182, filed on Nov. 21, 2013.

(51) Int. Cl. A61L 24/00; A61L 24/02; A61L 24/08

(52) U.S. Cl. CPC A61L 24/0036; A61L 24/08; A61L 24/02; A61L 24/0015; A61L 2430/02

(57) ABSTRACT

Macroporous cement compositions, methods of making the same, and methods of using the same, are described.

### RELATED APPLICATIONS

This application claims priority to U.S. Provisional Application Ser. No. 61/907,182, filed under 35 U.S.C. §111(b) on Nov. 21, 2013, the disclosure of which is incorporated herein by reference in its entirety.

### STATEMENT REGARDING FEDERALLY SPONSORED RESEARCH

This invention was made with no government support. The government has no rights in this invention.

### BACKGROUND OF THE INVENTION

Cancellous bone, also known as trabecular bone, is a type of osseus tissue that makes up bones. Compared to the other type of osseus tissue, called cortical bone or compact bone, cancellous bone is less dense and therefore has a higher surface area to mass ratio. While cortical bone is a dense collection of lamellar sheets composed of mineralized collagen fibers and surrounded by vascular channels, cancellous bone is a highly porous material with high surface area and low density (typically about 0.2-0.5 g/cm³). Cancellous bone has about 75-85% porosity with pores having diameters of about 200-700 μm, while cortical bone has only 5-10% porosity with pores having diameters of about 1-100 μm. Given this difference, cancellous bone is more suitable for metabolic activity, such as the exchange of cations, and is highly vascular. Cancellous bone is found at the ends of long bones, proximal to joints, and in the interior of vertebrae. It would be advantageous for a bone implant, such as a hardened cement mass, to mimic the porosity and pore size of cancellous bone.

Porosity and pore size in bone implants are important. The pores of a bone implant, such as a hardened cement mass, should have a diameter of at least 50 μm, preferably at least 100 μm, in order to promote ingrowth of bone into the implant. Methods currently known in the art for creating porosity in bone implants, such as freeze-casting of ceramics, require long wait times for the sintering process. There is a need in the art for an injectable cement composition with macroporosity that can be produced in real-time, at the site and time of the desired application, rather than having to wait for a sintering process to create macroporosity. There is a need to develop these granules in a time-effective and cost-effective method.

The technology of producing granules has existed for several decades. Granules have been fabricated from various calcium phosphate phases such as hydroxylapatite (HA), tricalcium phosphate (TCP), calcium-deficient hydroxyapatite (CDHA), dibasic calcium phosphate dihydrate (DCPD), silicon-substituted apatite, and biphasic calcium phosphates. While some efforts have utilized coral-derived granules or bovine trabecular materials, conventional methods involve preparation of slurries of chemical origin of various phases of calcium phosphates, followed by granulation and final sintering. Due to immunological issues associated with allografts, it is preferred that artificial chemicals are used for the manufacturing of these granules. The drawbacks of the conventional processes include very little presence of pores, or too small of granules. To increase the pore content, polymers are sometimes added, which requires high temperature burn-off, followed by sintering. During the burn-off, the polymers melt and may not retain their original shape and size. The results of such methods produce granules of high porosity content, up to 75%, with pore sizes as large as 400 μm. Introduction of polymers has thus become important, but not necessarily yielding the best results.

It is apparent that several criteria need to be met for the successful development of porous granules. First, an effective porogen should be chosen, preferably in a particular form, that can be cleanly removed from the system, while leaving the shapes of pores intact, without collapsing the surrounding struts around them. Second, the granules should be of relevant size, such 1 mm-2 mm in orthopedic applications and 0.25 mm-1 mm in periodontal surgery applications. Third, the process should be simple, preferably involving a single step. Fourth, it is preferable that no high temperature sintering is needed in order to simplify the process, while simultaneously addressing contamination-related issues. Fifth, it is also preferable that there is enough radio-opacity present. Finally, the handling strength should be such that the granules do not crumble either during the processing or when exposed to body fluids.

Recent developments in this field include the use of sodium chloride (NaCl) as a porogen because NaCl is both leachable and biocompatible. In fact, the use of NaCl as a porogen is a well known concept in fabricating biopolymeric scaffolds, such as polycaprolactone, gelatin, polyurethane, and polylactic-co-glycolic acid (PLGA). NaCl has also been used as a porogen in the production of granules in orthopedic cement technology. The resulting granules have poorly crystallized apatite as the predominant phase, with the granule being in the size range of 2 mm-4 mm, containing 50% porosity, and having pore sizes between 50 μm-550 μm. While there have been recent attempts to improve the technique, no new progress has been made to create granules of the correct sizes containing macropores of desired dimensions and fabricated at room temperature without the use of high temperature sintering. Thus, there remains a need in the art for new macroporous cement compositions, and new methods of making macroporous cement compositions.

### SUMMARY OF THE INVENTION

Provided herein is a macroporous cement composition comprising pores made from a hydrogen-evolving process that occurs in a solution containing a mixture of a post-microwaved powder and a liquid, where the pores range in size from about 100 μm to about 1 mm In certain embodiments, the hydrogen-evolving process is a reaction between a metal and water present in the liquid. In particular embodiments, the metal is selected from the group consisting of magnesium, iron, and aluminum. In particular embodiments, the metal is magnesium. In particular embodiments, the magnesium is in the form of spherical particles. In particular embodiments, the magnesium particles have a diameter of about 45 μm. In particular embodiments, the magnesium is present in an alloy with one or more of Al, Zn, or a rare-earth metal. In certain embodiments, the liquid further comprises silica. In certain embodiments, the macroporous cement composition further includes a biopolymer or polysaccharide. In particular embodiments, the macroporous cement composition includes alginate. In particular embodiments, the macroporous cement composition includes chitosan.

In certain embodiments, the post-microwaved powder comprises calcium phosphate that has been irradiated by microwaves. In certain embodiments, the macroporous cement composition comprises a magnesium phosphate. In certain embodiments, the composition comprises a strontium-doped calcium phosphate or a strontium-doped magnesium phosphate. In certain embodiments, the macroporous cement composition is injectable during the hydrogen-evolving process.

In certain embodiments, the macroporous cement composition further includes drug molecules in the pores. In certain embodiments, the macroporous cement composition further includes an antibiotic. In certain embodiments, the antibiotic is selected from the group consisting of clindamycin, tigecycline, vancomycin, ciprofloxacin, ofloxacin, sulfamethoxazole, trimethoprim/sulfamethoxazole, amoxicillin, penicillin V, penicillin G, procaine penicillin, benzathine penicillin, carbencillin, mezlocillin, ampicillin, piperacillin, bacampicillin, tiearcillin, ticarcillin, piperacillin/tazobactam, aztreonam, cefotetan, loracarbef, mefoxin, merrem, levofloxacin, lomefioxacin, primaxim, cycloserine, kanamycin, dicloxacillin, demeclocycline, minocycline, doxycycline, oxytetracycline, tobramycin, gentamicin, neomycin, amikacin, craramyein, nebcin, erythromycin/sulfisoxazole, netromycin, streptomycin, tobramycin, cefotaxime, cefuroxime, cefazoline, ceffibuten, ceffizoxime, cefaclor, cefopoerazone, cefprozil, cefadroxil monohydrate, ceftazidime, trimethoprim/sulfamethoxazole, cephalexin, cefazolin, cefamandole nafate, cefepime, cefonicid, sulfadiazine, norfloxacin, enoxacin, cefdinir, seromycin, ceftriaxone, cefixime, ceftazidime, clarithromycin, dirithromycin, methenamine, ethionamide, trovafioxacin, sparfloxacin, interfon-α, indinavir, ganciclovir, foscamet, lamivudine, famciclovir, rimantadine, zalcitabine, interferon-β, indinavir, ganciclovir, saquinavir, ritonavir, ribavirin, erythromycin, troleandomycin, azithromycin, eliiidamycin, colistin, amphotericin B, flucytosine, fluconazole, griseofulvin, grepafloxacin, ultramicrosize griseofulvin, terbinafine, ketoconazole, clotrimazole, dapsone, delavirdine, ziduvudine, amantadine, palivizumab, valacyclovir, didanosine, nelfinavir, nevirapine, ribavirin, cidofovir, pyrimethamine, metronidazole, furazolidone, atovaquone, stavudine, lamiduvine, acyclovir, mionazole, nystatin, itraconazole, chloroquine, pyrimethamine, mefloquine, hydroxychloroquine, capreomycin, permethrin, crotamiton, lindane, fluoro-uracil, ethambutol, rifabutin, isoniazid, aminosalicyclic acid, rifapentine, pyrazinamide, coenzoyl peroxide, chlorhexidine gluconate, sodium oxychlorosene, benzoyl peroxide, rifampin, rifampin/isoniazid, rifampin/isoniazid/pyrazinamide, nitrofurantoin, linezolid, nitrofurantoin, fosfomycin, nalidixic acid, atropine, oxytetracycline/sulfamethizole/phenazopyridine, mupirocin, chloramphenical, neomycin/polymyxin, tfimetorpim/polythyxin, tobramycin/dexamethasone, vidatabine, ciprofloxacin, ofioxacin, sulfacetamide, mupirocin, povidoneodine, gentamicin, nystatin, chloramphenicol, bacitracin, sulconazole, terbinafine, tetrachlorosalicylanilide, metronidazole, metromdazole, ciclopiroxolamine, clotrimazole, clotrimazole/betamethasone, butenafine, clotrimazole, nattifine, oxiconazole, selenium, econazole, penciclovir, and pharmaceutically acceptable salts thereof.

In certain embodiments, the macroporous cement composition further includes a growth factor. In particular embodiments, the growth factor is selected from the group consisting of BMP1, BMP2, BMP3, BMP4, BMP5, BMP6, BMP7, BMP8a, BMP8b, BMP10, or BMP15.

Further provided is a cement composition comprising a powder component, a liquid component, and a porogen capable of reacting with the liquid component to produce a gas, wherein the powder component is mixed with the porogen and the liquid component to form a gaseous solution, and the gaseous solution sets to form a hardened porous mass.

Further provided is a cement composition convertible by bodily processes into a macroporous cement composition as described herein.

Further provided is an injectable cement composition comprising macropores.

Further provided is a gaseous solution comprising hydrogen bubbles and spherical magnesium particles.

Further provided is a method for making a cement composition, the method including the steps of mixing a powder component with a porogen and a liquid component to form a gaseous solution, wherein the porogen reacts with water in the liquid component to produce hydrogen gas; and allowing the gaseous solution to set into a hardened porous mass, wherein the hydrogen gas bubbles create pores in the hardened mass.

Further provided is a method for making a cement composition, the method including the steps of mixing a powder component with a setting solution to form a paste; irradiating the paste with microwaves to form post-microwaved powder; mixing the post-microwaved powder with magnesium particles and a liquid to produce hydrogen gas bubbles in a gaseous solution; and allowing the gaseous solution to set into a hardened porous mass, wherein the hydrogen gas bubbles create pores in the hardened porous mass. In certain embodiments, the second setting solution includes silica.

In certain embodiments of the methods described herein, the setting solution comprises at least one of alginate or chitosan. In certain embodiments, the hardening of the gaseous solution occurs simultaneously with the production of hydrogen bubbles. In certain embodiments, the cement composition is extruded through a syringe while pores are forming from the hydrogen bubbles.

Further provided is the product of any method described herein.

Further provided is a bone implant comprising a macroporous cement composition described herein.

Further provided is a kit for making a macroporous cement composition, the kit comprising a first container housing a post-microwaved powder and a porogen, and a second container housing a liquid. In certain embodiments, the kit further includes a syringe.

Various aspects of this invention will become apparent to those skilled in the art from the following detailed description of the preferred embodiment.

### BRIEF DESCRIPTION OF THE DRAWING

The patent or application file contains one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1B: Photographs of a macroporous cement composition hardening in a test tube.

FIGS. 2A-2F: Photographs of hardened cement granules with macro-sized pores visible.

FIG. 3: X-ray diffraction pattern of a macroporous cement composition.

FIGS. 4A-4J: SEM images of hardened macroporous cement compositions. Various sizes of pores are seen with the aid of the scale provided in each image.

### DETAILED DESCRIPTION OF THE INVENTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

The present disclosure relates to a one-step process for making macroporous cement compositions, such as those from alkaline earth phosphates, or granules with macroporosity for orthopedics and dental applications. Provided herein are compositions, and methods for making such compositions, involving the use of a gas-evolving process to create porosity in a granule. In accordance with the present disclosure, granules containing macropores are produced using a particular type of porogen in any suitable cement composition. This disclosure is particularly useful with, though by no means limited to, the bone cement compositions disclosed in PCT Publication No. WO 2013/116457 A1 dated Aug. 8, 2013, which is PCT application PCT/US13/24040 filed on Jan. 31, 2013, entitled “Injectable, Biodegradable Bone Cements And Methods Of Making and Using Same.” The entire disclosure of WO 2013/116457 A1 is incorporated herein by reference. However, the referenced PCT publication does not describe a process for making granules with macropores. The ability to produce granules with macropores is important in both orthopedics and in dentistry. Granules having pore sizes similar to those in human bones can act as three-dimensional scaffolds for cell delivery in tissue engineering, and are also effective in drug and growth factor delivery.

This disclosure represents improvements for producing granules containing macropores using various cement technology and compositions. Though calcium phosphate or magnesium phosphate cements, such as those disclosed in WO 2013/116457 A1, are often mentioned for illustrative purposes, it is to be understood that a gas-evolving process can be used to create macroporosity in any cement composition that involves the use of a liquid. Therefore, suitable final cement compositions include, but are not limited to, Ca-P cements such as hydroxyapatite [Ca₁₀(PO₄)₆(OH)₂], tetracalcium phosphate (TTCP, Ca₄(PO₄)₂O), tricalcium phosphate [α-TCP, α-Ca₂(PO₄)₂ and β-TCP, β-Ca₃(PO₄)₂], dicalcium phosphate anhydrous (DCPA, monetite, CaHPO₄), di-calcium phosphate dehydrate (DCPD, brushite, CaHPO₄·2H₂O), octacalcium phosphate (OCP, Ca₈H₂(PO₄)₆·5H₂O); Mg-P cements such as MgNH₄PO₄, formed via reaction between acid part (ammonia salts) and base part (MgO or Mg(OH)₂), struvite, newberyite, or cattiite; Sr—P cements (SPCs), such as Ca—P cements doped with strontium, or Mg—P cements doped with strontium; calcium sulfate cements, such as calcium sulfate hemihydrate (CSH) and calcium sulfate dehydrate (CSD); poly methyl methacrylate (PMMA) cements; and polyacrylic acid (PAA) cements.

Existing porous cement compositions, which typically use NaCl as a porogen, ordinarily require high temperature sintering steps from granulation to leaching of NaCl. Even if high temperature sintering is not required, the known methods typically require a few steps from granulation to leaching. Furthermore, these processes are not continuous because of the steps involved in creating the porosity. The known methods are specifically tailored toward the fabrication of granules instead of an injectable product, despite the appeal of an injectable macroporous cement composition. The known porous cement products are also not radio-opaque. Thus, the methods and compositions disclosed herein include many advancements and improvements over currently known methods and cement compositions.

In accordance with the present disclosure, the cements described herein use a porogen, which instantaneously creates porosity and is biocompatible. This porogen does not require subsequent leaching in water, unlike cements that use NaCl as a porogen. Furthermore, because of the instantaneous creation of pores, the process can be truly termed a single-step process. Either continuous or batch processes can be used to make these cements, and the cements can be made into injectable compositions. The ability to make an injectable, macroporous cement composition in real-time (that is, within a matter of a few minutes) is a significant advantage over existing methods and cement compositions. Furthermore, the cement compositions described herein generate little or no exothermicity during hardening. This alleviates concerns of possible damage to surrounding tissue while curing. The compositions are also pH-balanced.

The particular porogen used in this disclosure is one that reacts with the liquid component to give off a gas. The gas bubbles produced from this reaction do not disappear prior to the gaseous solution hardening. Rather, as the gas bubbles attempt to escape, they are trapped by the hardened of the solution, thereby creating pores in the hardened cement mass when the gaseous solution sets. In preferred embodiments, the porogen is one that reacts with water to give off hydrogen gas. However, other gases can be generated to create porosity. Suitable such gases include, but are not limited to, hydrogen, carbon dioxide, oxygen, nitrogen, argon, helium, carbon monoxide, methane, propane, and combinations thereof.

In particular embodiments, the porogen is magnesium metal particles. However, there are many reactants which produce hydrogen gas upon mixing with water, and any such reactant is useable as a porogen. Those skilled in the art will recognize that metals other than magnesium, such as most alkali metals and alkaline earth metals, can also react with water to produce hydrogen gas. In particular, metals such as iron, aluminum, sodium, lithium, potassium, rubidium, caesium, francium, calcium, strontium, barium, and uranium are suitable non-magnesium metals that are also useful as a porogen in certain embodiments of the methods and cement compositions described herein. Also suitable are alkaline earth metal hydrides, which react with water to produce hydrogen gas. Moreover, many alloys, such as, but not limited to, alloys of magnesium and yttrium, magnesium and zinc, magnesium and aluminum, or magnesium and other rare-earth metals, can produce hydrogen gas upon mixture with water and thus are suitable for use as a porogen in the methods and compositions described herein. However, magnesium is described for exemplary purposes because magnesium corrodes quickly and forms bubbles upon mixing with water quickly. Magnesium also has a cytotoxicity better suited for cement applications than other metals.

Magnesium is known to be highly prone to corrosion and is readily available in powder form. Magnesium is the fourth most abundant element in the mammalian body, and most of it is stored in the skeleton. When magnesium comes into contact with water, it evolves hydrogen gas. Thus, when magnesium particles are mixed with a powder component and an aqueous liquid, the evolution of hydrogen bubbles creates instant porosities of up to about 1000 μm. Without limitation, the pores created by evolving hydrogen gas range from about 100 μm to about 1 mm in size. By carefully changing the power-to-liquid ratio, it is possible to inject this free-flowing mass as a porous product containing macroporosity.

The magnesium used as a porogen is preferably in the form of spherical particles in a very fine magnesium mesh. The reason spherical particles are preferred is because they have a higher surface area, and therefore greater reactivity. The greater reactivity allows for more hydrogen bubbles to form from the reaction between magnesium and water. Additionally, the shape of the particles determines the shape of the pores. Therefore, spherical magnesium particles produce spherical pores in the resulting cement. If differently shaped pores are desired for a particular cement application, the shape of the porogen particles can be chosen accordingly. The magnesium particles can also be sieved into different size fractions in order to control the porosity of the resulting cement.

A typical bone cement is made by mixing a basic powder component with an acidic liquid component, referred to as a setting solution, in order to form a paste, resulting from an acid-base reaction, that sets into a hardened mass. Many variations of this method have been developed. For instance, as described in WO 2013/116457, the paste can be subjected to microwave radiation prior to hardening and crushed up, in order to form dry powder, also referred to as post-microwaved powder (PMP), that is then mixed with a second setting solution to form a radiation-assisted paste that sets into a hardened mass. Silica can be included in the second setting solution in order to improve the strength of the resulting cement. In a typical example, the second setting solution can contain about 40% silica suspended in water. Also described in WO 2013/116457, a setting solution can include a biopolymer, such as chitosan, in order to improve the strength of the resulting cement. Alternatively, a polysaccharide, such as alginate, can be included in a setting solution in order to improve the handling of the cement. Alginate dissolves in the body quickly, and is very hydrophilic. The inclusion of alginate tends to improve the handling properties of the resulting cement. Furthermore, the addition of magnesium oxide can improve the strength of the resulting cement. These methods of producing a bone cement are referred to for exemplary purposes herein because they are especially conducive to preparing an injectable, macroporous composition in a matter of a few minutes, though any other method of producing a cement composition that involves the use of a liquid may be used. In preferred embodiments, the liquid is an aqueous solution. This is because the chemistry of forming hydrogen gas from a reaction between a porogen and water is especially useful for creating injectable, macroporous compositions.

In any method of making the macroporous cement compositions, the porogen particles are added either to the powder to be mixed with a liquid (i.e., a setting solution or second setting solution), to a pre-mixed paste that ultimately sets into a hardened mass, to the liquid immediately prior to mixing with the powder component, or to the powder and liquid simultaneously. Upon contact with the liquid, the porogen begins to react, for instance with water, to form gas bubbles, such as hydrogen gas bubbles. The gas bubbles try to escape from the gaseous solution while the solution sets, but the gaseous solution sets into a hardened mass before the gas bubbles can fully escape. In this manner, the gas bubbles become pores in the resulting hardened mass, thereby creating a porous cement mass. Given the size of the pores possible using this method, the cement mass is a macroporous cement mass. Those skilled in the art will understand that the injectable cement can be manipulated, shaped, or extruded while setting in order to form a desired shape, for example to fit into a desired mold or position. The gaseous solution can be formed into macroporous blocks or putties before hardening.

The setting time for a cement composition using magnesium can vary widely, and can be customized In certain embodiments, the setting time is between about 30 seconds and 1 minute. This period of time allows for hydrogen bubbles to come to the surface of the composition. In addition, the pore attributes can be altered and customized for a particular application. For instance, by incorporating alloys of magnesium, such as with Al, Zn, or rare-earths, the degradation rate of the metallic additions, and therefore the hydrogen evolution rates, is changed. This, in turn, affects the amount and size of pores created before the composition sets into hardened granules.

Given that magnesium is a brittle ceramic, there is a possibility of the granules crumbling if too many pores or too large of pores are generated. To avoid this, the cement composition should be made with sufficient strength. Thus, instead of granulating batches, a continuous process of injection of the cement pase involving extrusion followed by subsequent chopping can be a method for production. A variation of the process may involve injecting the high porosity mixture directly into an orthopedic injury or cavity site. During the process of extrusion/injection, substantially all the magnesium powder particles totally convert into hydrogen gas bubbles. By carefully choosing the size and distribution of magnesium particles, the ranges of pore sizes can be easily controlled.

The powder-to-liquid ratio determines the setting time and hardnening of the cement composition. Thus, the setting time of a macroporous cement composition can be adjusted by changing the powder-to-liquid ratio of the cement. In this manner, the cement compositions are customizable for a desired application. For instance, the relevant practitioner would likely want the setting time of a cement to be used for filling a cavity in a tooth to be somewhat quicker than the setting time of a cement to be used in an orthopedic surgery application.

If a reduced particle size of a particular cement composition is desired for a certain application, such particle reduction can be accomplished by using, for example, an agate pestle and mortar, a ball mill, a roller mill, a centrifugal-impact mill and sieve, a cutter mill, an attrition mill, a chaser mill, a fluid-energy mill, and/or a centrifugal-impact pulverizer. Particle size reduction may be desired for treating bone defects through a method that involves breaking up the hardened cement into pellets and filling a hole or cavity in a bone with the pellets.

The cement compositions described herein can be used to deliver one or more agents. Suitable agents include, but are not limited to: drugs, such as bisphosphonates, parathyroid hormones, antiseptics, anti-inflammatories, anti-infectives, antibiotics, antifungals, bone resorption inhibitors, antivirals, analgesics, chemotherapeutic agents, anticoagulants, antihistamines, antineoplastic agents, steroids, and vitamins; proteins, including growth factors, such as the bone morphogenetic proteins BMP1, BMP2, BMP3, BMP4, BMP5, BMP6, BMP7, BMP8a, BMP8b, BMP10, or BMP15; cells, such as mesenchymal stem cells; nucleic acids, such as microRNAs, antisense microRNAs, siRNAs, shRNAs, cDNA, and aptamers; drug delivery vehicles (whether loaded with drugs or not), such as liposomal formulations or carbon nanotubes; diagnostic agents; materials designed to release ions, such as silver ions in the form of a colloidal silver; bioactive glass; and combinations thereof. The agents can be incorporated into the walls of the pores in the macroporous cement, and are released upon degradation of the cement.

The cement compositions disclosed herein are particularly useful for drug delivery. The macroporous cements described herein have increased surface area from which drugs can be delivered. To load a drug in the cement, the drug can be dissolved in a gaseous solution prior to setting into a macroporous cement mass, or a macroporous cement composition (before or after it sets) can be soaked in a solution or suspension comprising the drug, before the composition is injected or placed into or onto an anatomical location. The drug can then be released into the subject from the cement matrix. Embodiments resulting in sustained release of drugs are also possible. The rate of release of a drug depends on the degradability of the cement. Thus, the degradability of the cement composition can be controlled (for instance, by changing the powder and liquid components in order to produce a different phase) in order to control the rate at which a drug is released from the cement composition. Alternatively, sustained release can be accomplished by coating the cement matrix with polymers including PLA/PGA, polyacrylic acid, hydroxyl methylcellulose, and/or chitosan. As yet another alternative, the drugs may be in the form of a lipophilic ester, such as an acyl derivative (for instance, an acetyl ester), so as to allow release of the drugs from the cement over a prolonged period of time as a result of esterase activity in the physiological fluids contacting the cement in the body.

The delivery of drugs from the cement is dictated by the composition of the cement. The release of drugs depends upon the rate of degradation of the composition. Thus, cement compositions that degrade faster will release drugs at a quicker rate. For instance, DCPD cement is somewhat faster at degrading, and therefore faster at releasing drugs, than monetite cement.

In one particular embodiment, the cement composition is useful for treating or preventing an infection, and the composition includes at least one antibiotic. Suitable antibiotics include, but are not limited to, penicillins, tetracyclines, glycylcyclines, macrolides, sulfonamides, cephalosporins, monobactams, carbapenems, aminoglycosides, quinolones, oxazolidinones, and combinations thereof. In certain non-limiting examples, the composition includes at least one antibiotic selected from the group consisting of clindamycin, tigecycline, vancomycin, ciprofloxacin, ofloxacin, sulfamethoxazole, trimethoprim/sulfamethoxazole, amoxicillin, penicillin V, penicillin G, procaine penicillin, benzathine penicillin, carbencillin, mezlocillin, ampicillin, piperacillin, bacampicillin, tiearcillin, ticarcillin, piperacillin/tazobactam, aztreonam, cefotetan, loracarbef, mefoxin, merrem, levofloxacin, lomefioxacin, primaxim, cycloserine, kanamycin, dicloxacillin, demeclocycline, minocycline, doxycycline, oxytetracycline, tobramycin, gentamicin, neomycin, amikacin, craramyein, nebcin, erythromycin/sulfisoxazole, netromycin, streptomycin, tobramycin, cefotaxime, cefuroxime, cefazoline, ceffibuten, ceffizoxime, cefaclor, cefopoerazone, cefprozil, cefadroxil monohydrate, ceftazidime, trimethoprim/sulfamethoxazole, cephalexin, cefazolin, cefamandole nafate, cefepime, cefonicid, sulfadiazine, norfloxacin, enoxacin, cefdinir, seromycin, ceftriaxone, cefixime, ceftazidime, clarithromycin, dirithromycin, methenamine, ethionamide, trovafioxacin, sparfloxacin, interfon-α, indinavir, ganciclovir, foscamet, lamivudine, famciclovir, rimantadine, zalcitabine, interferon-β, indinavir, ganciclovir, saquinavir, ritonavir, ribavirin, erythromycin, troleandomycin, azithromycin, eliiidamycin, colistin, amphotericin B, flucytosine, fluconazole, griseofulvin, grepafloxacin, ultramicrosize griseofulvin, terbinafine, ketoconazole, clotrimazole, dapsone, delavirdine, ziduvudine, amantadine, palivizumab, valacyclovir, didanosine, nelfinavir, nevirapine, ribavirin, cidofovir, pyrimethamine, metronidazole, furazolidone, atovaquone, stavudine, lamiduvine, acyclovir, mionazole, nystatin, itraconazole, chloroquine, pyrimethamine, mefloquine, hydroxychloroquine, capreomycin, permethrin, crotamiton, lindane, fluoro-uracil, ethambutol, rifabutin, isoniazid, aminosalicyclic acid, rifapentine, pyrazinamide, coenzoyl peroxide, chlorhexidine gluconate, sodium oxychlorosene, benzoyl peroxide, rifampin, rifampin/isoniazid, rifampin/isoniazid/pyrazinamide, nitrofurantoin, linezolid, nitrofurantoin, fosfomycin, nalidixic acid, atropine, oxytetracycline/sulfamethizole/phenazopyridine, mupirocin, chloramphenical, neomycin/polymyxin, tfimetorpim/polythyxin, tobramycin/dexamethasone, vidatabine, ciprofloxacin, ofioxacin, sulfacetamide, mupirocin, povidoneodine, gentamicin, nystatin, chloramphenicol, bacitracin, sulconazole, terbinafine, tetrachlorosalicylanilide, metronidazole, metromdazole, ciclopiroxolamine, clotrimazole, clotrimazole/betamethasone, butenafine, clotrimazole, nattifine, oxiconazole, selenium, econazole, penciclovir, or a pharmaceutically acceptable salt thereof.

In another particular embodiment, the cement composition is particularly useful for the healing of a fracture, and the composition includes at least one of BMP2 or BMP7. In another particular embodiment, the cement composition is useful for the prevention or management of osteoporosis, and the composition includes a drug selected from the group consisting of risedronate, etidronate, alendronate, teriparatide, strontium ranelate, raloxifene, denosumab, and calcitonin

Various additives, other than drugs, may be included in any of the cement compositions described herein to adjust their properties and the properties of the hardened cements produced. Examples of suitable additives include, but are not limited to, proteins, osteoinductive and/or osteoconductive materials, X-ray opacifying agents such as strontium phosphate or strontium oxide, supporting or strengthening filler materials, crystal growth adjusters, viscosity modifiers, additional pore-forming agents, color change agents, immersing liquids, carboxylates, carboxylic acids, α-hydroxyl acids, metallic ions, or mixtures thereof. Other suitable additives include substances that adjust setting times (such as pyrophosphates or sulfates), increase injectability or cohesion (such as hydrophobic polymers like collagen), or alter swelling time.

In addition to delivering drugs or other agents, the macroporous cement compositions described herein can be used for any bone cement application. By way of non-limiting examples, the macroporous cement compositions can be: used as a bone filler in areas of the skeleton where bone may be deficient; used to fill, augment, and/or reconstruct maxillofacial osseous bone defects, including for periodontal, oral, and cranio-maxillofacial application; packed gently into bony voids or gaps of the skeletal system (i.e., the extremities, pelvis, and spine), including for use in postero-lateral spinal fusion or vertebral augmentation procedures with or without appropriate stabilizing hardware; used to fill defects which may be surgically creasted osseous defects or osseous defects created from traumatic injury to the bone; used to heal a bone fracture; used as a filling material in teeth; coated on titanium implants; and added to calcium sulfate dehydrate (CSD) cements in order to significantly increase various properties of the CSD cements. The macroporous cement compositions can provide a bone void filler that resorbs and is replaced by bone during the healing process.

The macroporous cement compositions can be supplied to the user in a variety of forms, including as a powder or powder mixture which is later mixed with a solvent to make a slurry or putty, or as a pre-mixed putty which may contain a non-aqueous extender such as, but not limited to, glycerine and/or propylene glycol. The pre-mixed putty would allow the cement to set upon contact with water. In such embodiments, the porogen can be added with the water in order to introduce porosity. Also, any of the cement compositions described herein can be delivered via injection. That is, a cement composition can be transferred to a syringe for injection into a mammalian body prior to fully hardening. Furthermore, the cement compositions may be supplied with, or in, the instrumentation which is used to introduce the cement into the body. Examples of such instrumentation include, but are not limited to, a syringe, a percutaneous device, a cannula, a biocompatible packet, a dentula, a reamer, a file, or other forms which will be apparent to those of ordinary skill in the art. It is also understood that the cement compositions described herein could be delivered into the body in such a form as to be converted by bodily processes into the macroporous cement compositions disclosed herein.

The cement compositions disclosed herein may also be made available to the user (including practitioners such as surgeons, veterinarians, or dentists) via a kit containing one or more key components. A non-limiting example of such a kit comprises a dry homogeneous powder (composed of the powder component and magnesium particles) in one container, and the liquid component in another container, where the containers may or may not be present in a combined configuration. Many other kits are possible, such as kits comprising a pre-mixed putty instead of the powder and setting solution, and kits including a syringe or multiple syringes for injecting a macroporous cement composition formed from the components of such kit. The kits typically further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a CD-ROM, flash drive, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

The components to form a macroporous cement composition as described herein may be present as a packaged element. The cements are generally provided or employed in a sterilized condition. Sterilization may be accomplished by several methods such as radiation sterilization (e.g., gamma-ray radiation), dry heat sterilization, or chemical cold sterilization.

### EXAMPLES

### Example 1

Pure magnesium powder (−60 mesh) was weighed out at 0.05 grams and added into a mortar bowl. Then, 0.05 grams of magnesium oxide with 2 grams of the microwave-irradiated CaP cement powder used PCT Publication WO 2013/116457 were added to the mortar bowl. The powders were mixed thoroughly using an agate pestle to provide a uniform reaction. Finally, 0.5 ml of water and 1.5 ml of silica sol were added to the mixture and manually mixed using an agate pestle. Use of the agate pestle was stopped after all the powder had come into contact with the water. This allowed for the reaction to occur and for hydrogen bubbles to escape, creating pores throughout the mass without any trace of magnesium left. After the reaction took place, the macroporous granules reached optimum strength in approximately two hours. The granules were then placed into a water bath. After twenty-four hours, the cement remained intact and retained its strength without getting crumbled into powder.

### Example 2

Post-microwaved powder (PMP) was synthesized from calcium hydroxide (98% Ca(OH)₂ Fisher, Fair Lawn, N.J. as the source of calcium along with the setting solution). The PMP was comprised of 18.4 g of Ca(OH)₂ and 4.6 g of Mg(OH)₂. This ratio can be altered in order to make various batch sizes of different yields. The setting solution was prepared by combining sodium bicarbonate (NaHCO₃, Spectrum, CAS: 144-55-8), de-ionized water, and phosphoric acid (H₃PO₄, Spectrum, CAS: 7664-38-2). To yield 180 ml of complete setting solution, the setting solution consisted of 24 g sodium bicarbonate, 24 ml DI water, and 156 ml phosphoric acid, respectively. PMP was prepared by manually mixing calcium hydroxide with the setting solution using a mortar and pestle. For example, a batch of PMP was made by dispersing 18.4 g of calcium hydroxide and 4.6 g of magnesium hydroxide in 30 ml of setting solution, and completely mixing the powder in the setting solution through mixing for one minute. The resulting paste was then microwaved for nine minutes with 120 watts power to dehydrate the cement in order to stop the reaction. The resulting hard paste was crushed with a mortar and pestle and finally ground to create a fine powder. This PMP powder was used in the subsequent examples to create macroporous compositions.

### Example 3

The as-synthesized PMP was mixed with spherical Mg particles (<325 mesh; on the average ˜45 μm in diameter) in the compositions detailed in Table 1, below.

TABLE 1
PMP Compositions with Mg Particles

| Mg powder (g) | MgO (g) | PMP (g) | H₂O (ml) |
|---|---|---|---|
| 0.05 | 0.05 | 2 | 1 |
| 0.05 | 0.1 | 2 | 1.1 |

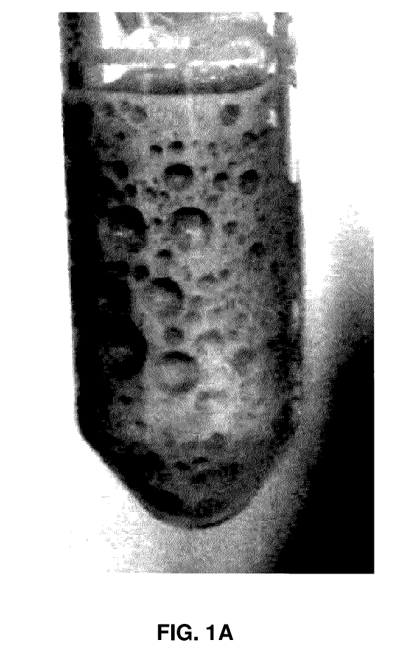

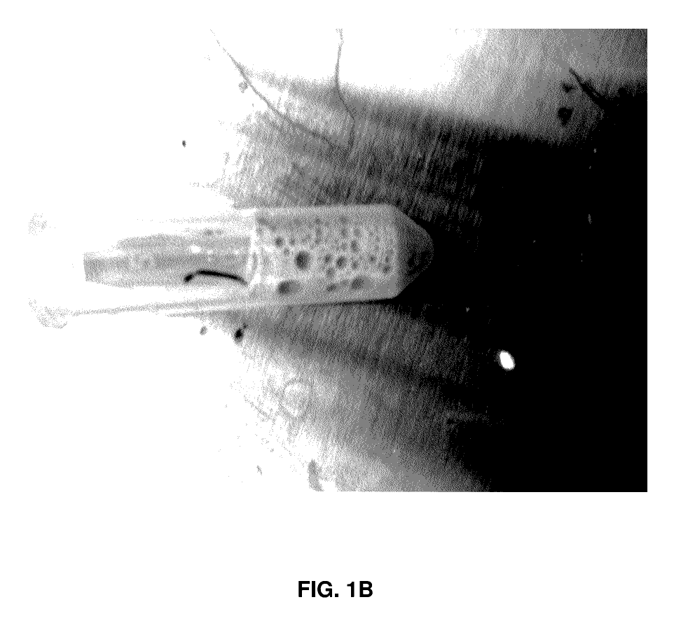

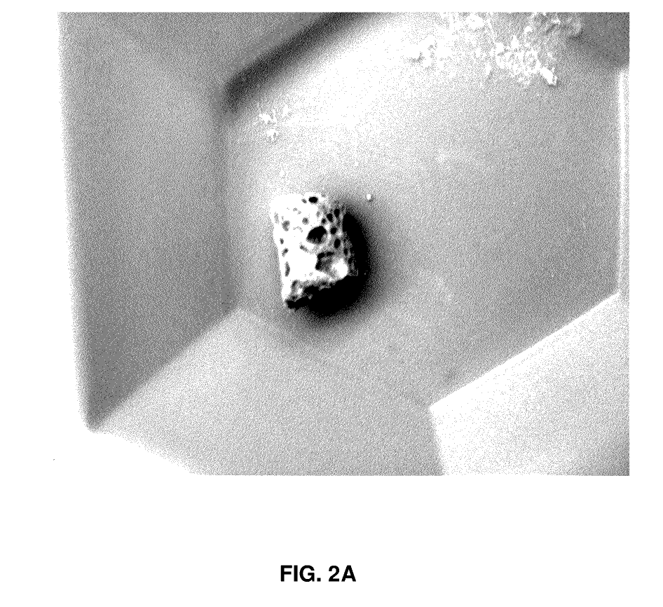

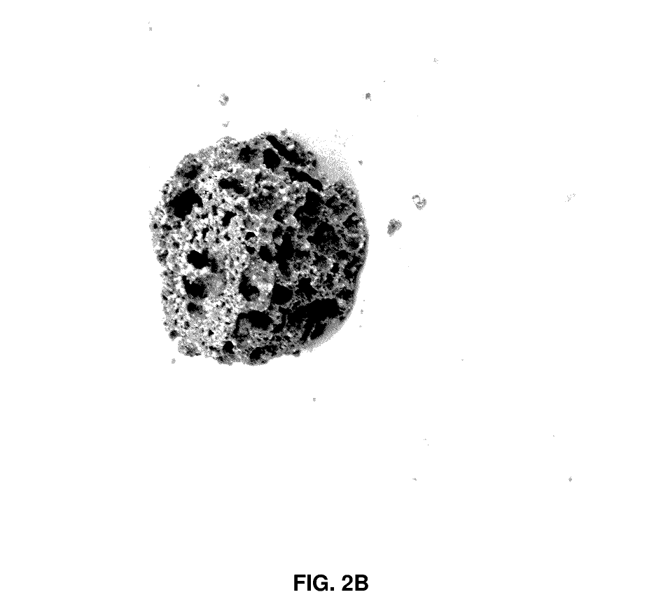

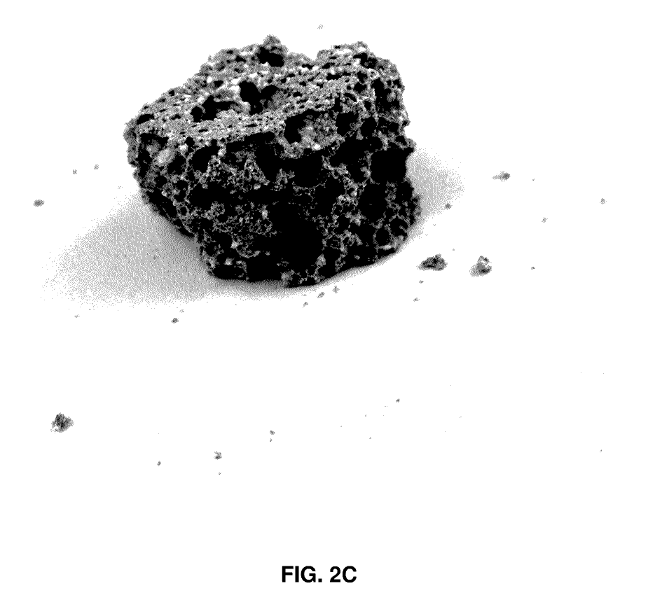

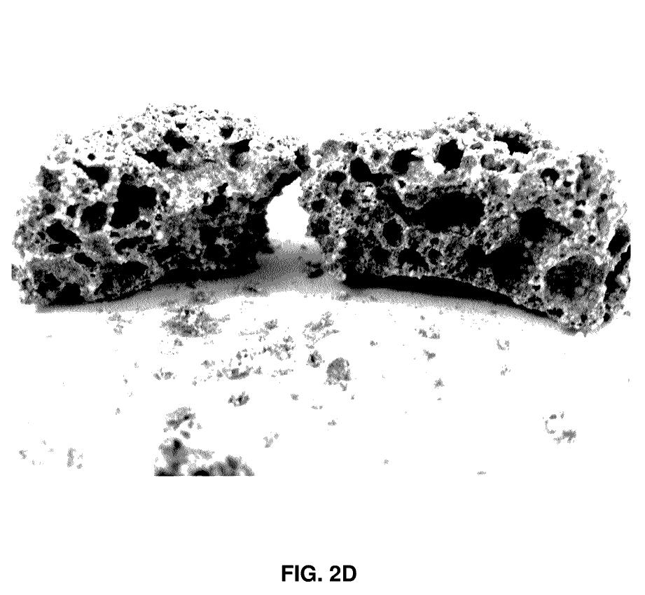

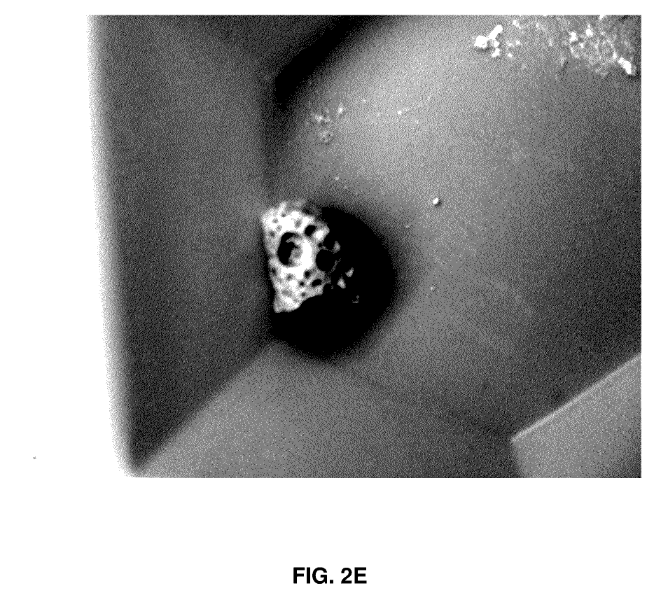

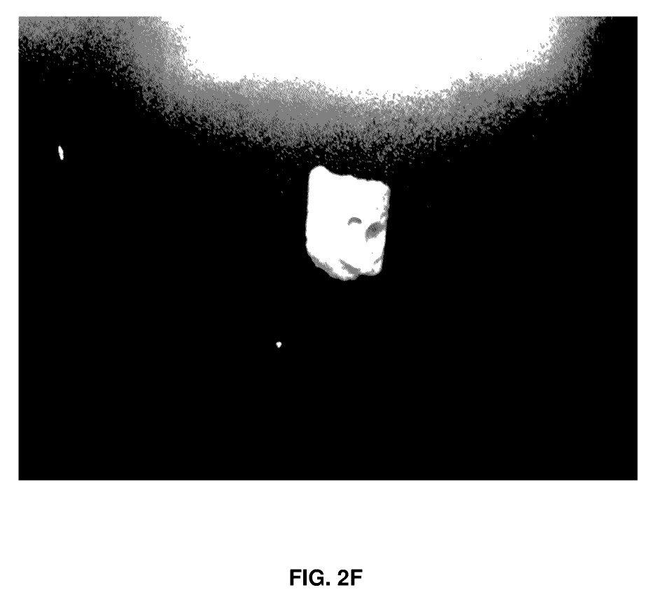

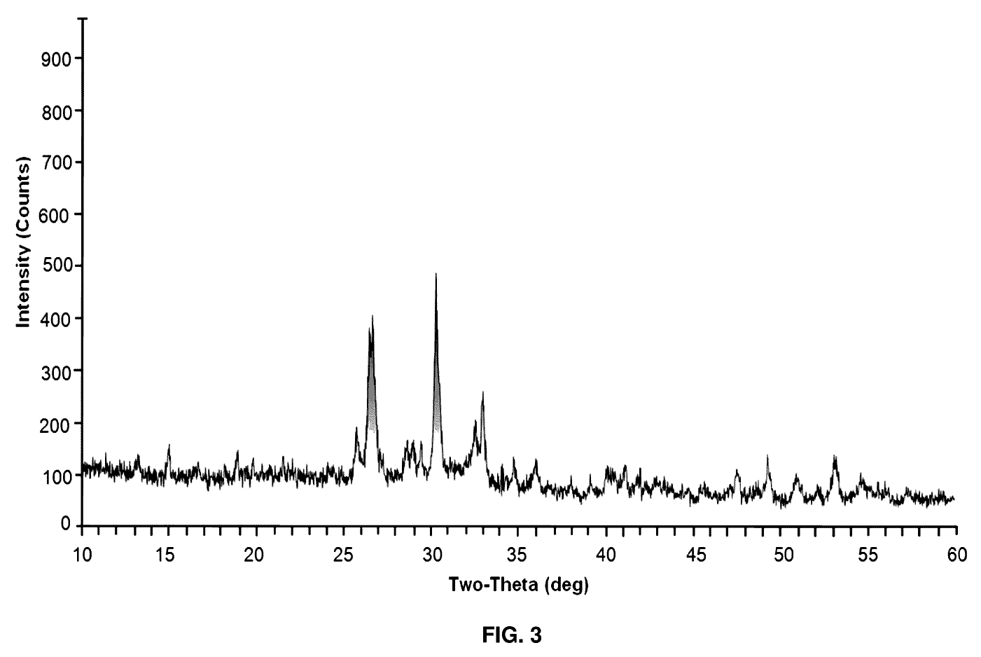

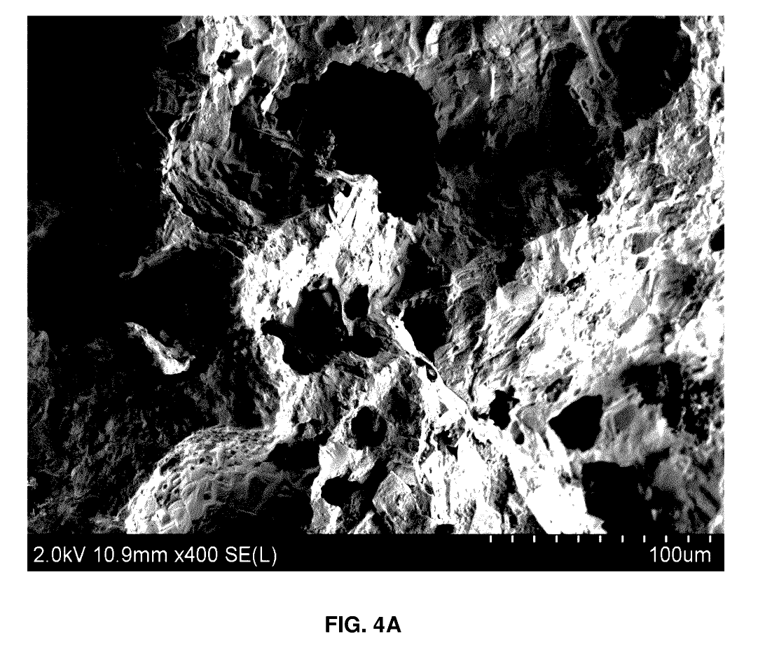

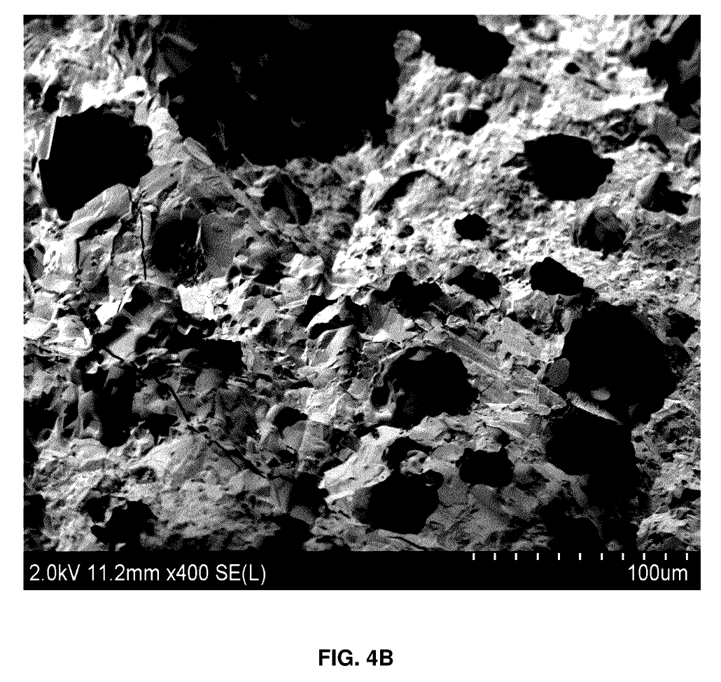

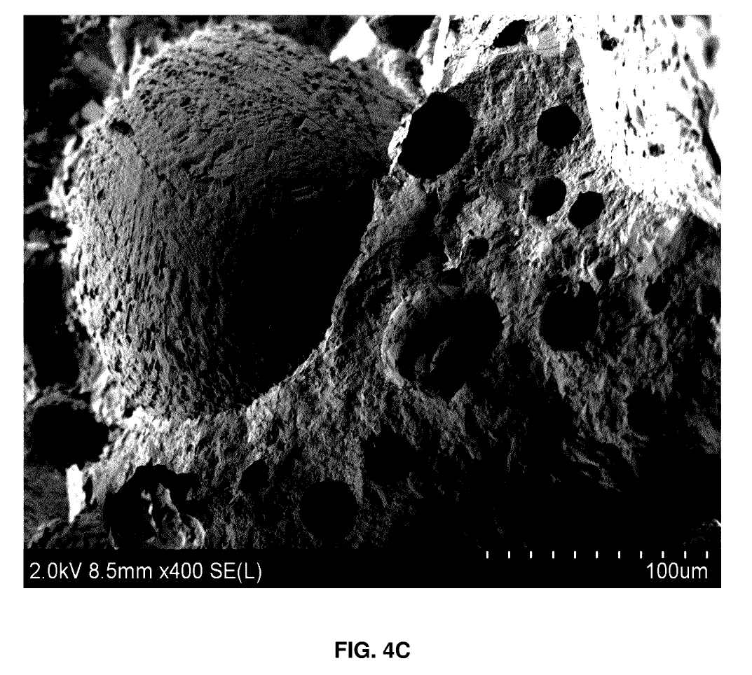

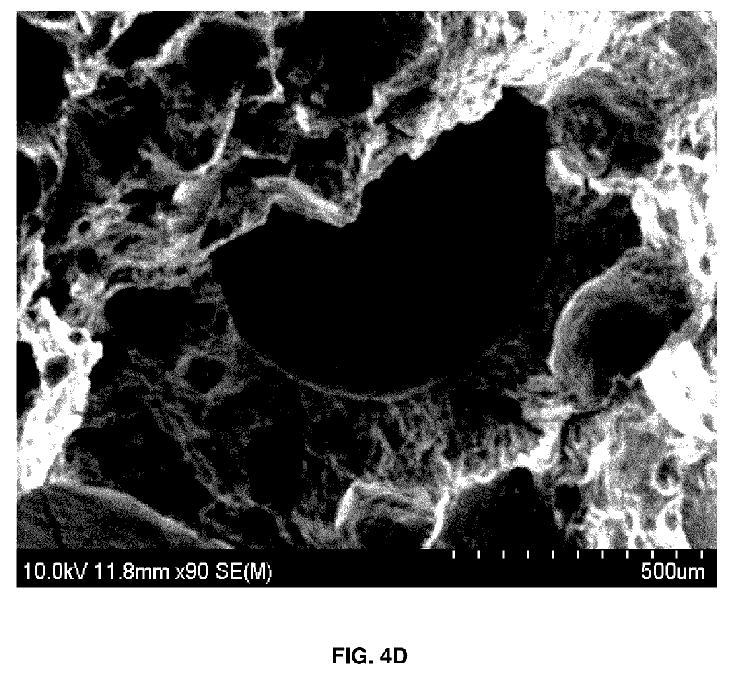

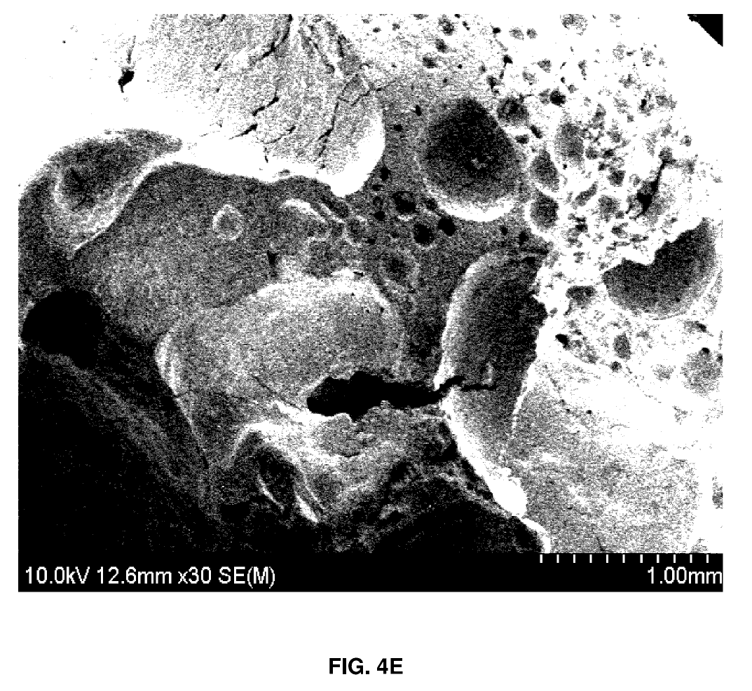

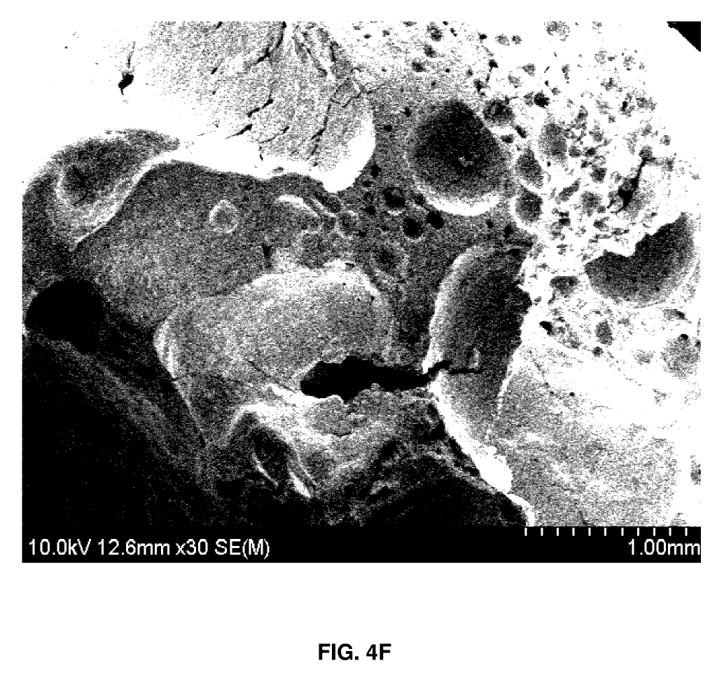

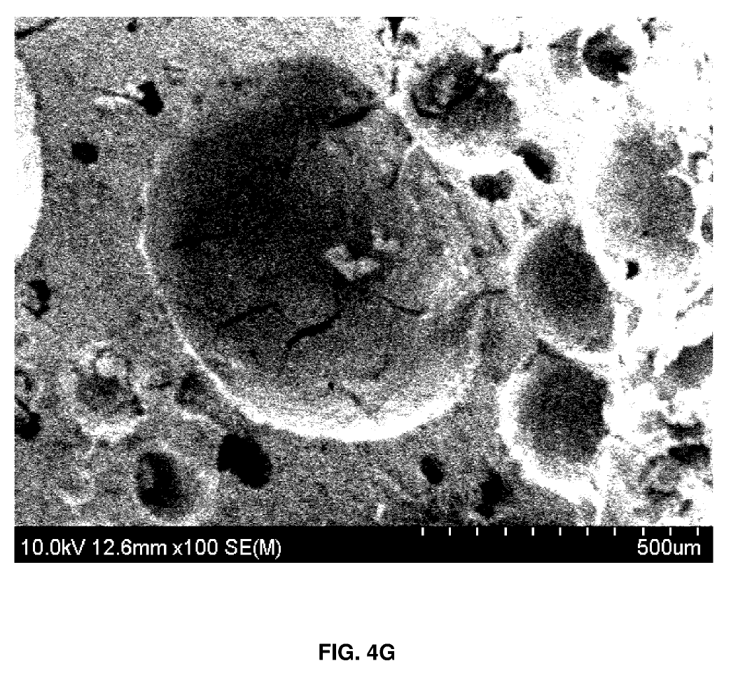

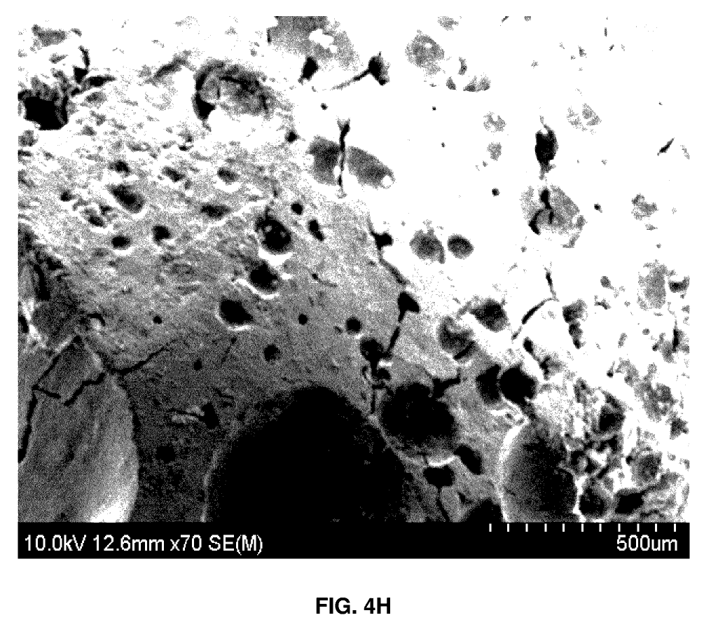

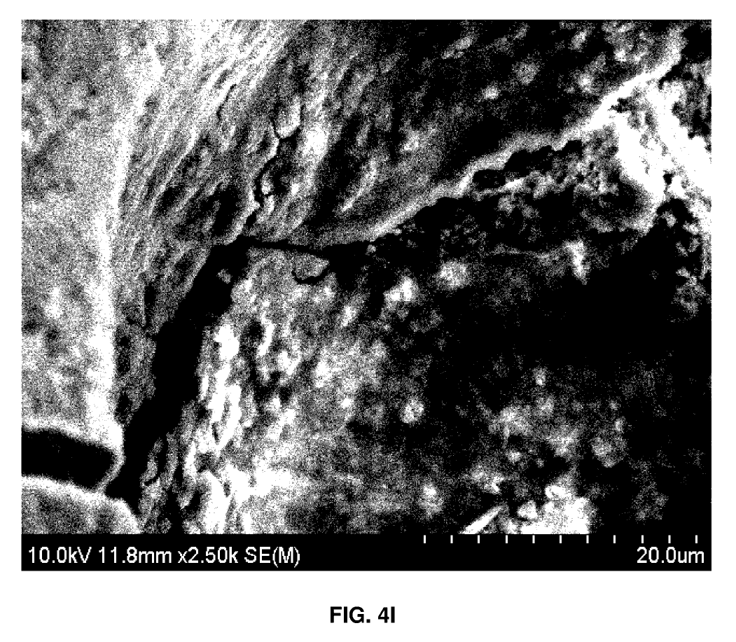

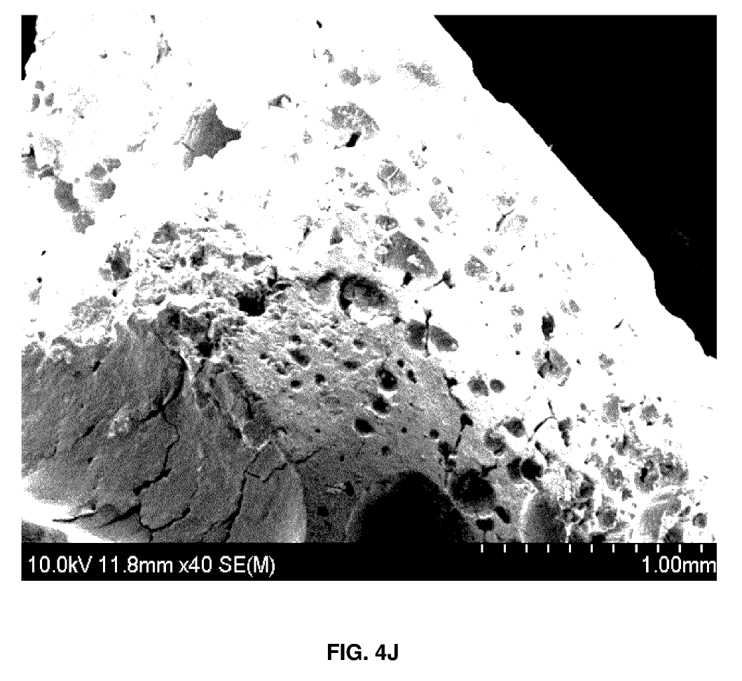

Both of the above compositions were gently mixed in a mortar and pestle. When the Mg particles were dissolved, there was concomitant evolution of H₂ gas. Within 2-3 minutes, the compositions began to harden while creating macropores approximately 500 μm in diameter. The formation of pores from hydrogen gas evolution can be seen in the photographs in FIGS. 1A-1B. These masses could be injected into a thin tube with pores still evolving in situ, prior to hardening. After complete hardening, the granules had sufficient handling strength. Photographs of the resulting hardened granules are shown in FIGS. 2A-2F. An X-ray powder diffraction pattern of a macroporous composition made by this process is shown in FIG. 3. FIGS. 4A-4J are SEM images of a hardened granule from this process. The SEM images show the different sizes of the pores formed in the hardened granules.

### Example 4

The procedure of Example 3 was repeated with a slightly different composition, as noted in Table 2, below.

TABLE 2
PMP Composition with Mg Particles and Higher Volume of Water

| Mg powder (g) | PMP (g) | H₂O (ml) |
|---|---|---|
| 0.1 | 2 | 2 |

This composition was made the same way as described in above, and pore generation took place in situ, as described in the previous example. With the addition of more water, the setting time increased. However, the strength was reduced due to the more time available for the macropores to grow.

### Example 5

To enhance the strength of the macroporous samples, both MgO and colloidal silica (HS-40, WR Grace) were incorporated into the sample. This composition is detailed in Table 3, below.

TABLE 3
PMP Composition with Colloidal Silica and MgO

| Mg powder (g) | MgO (g) | PMP (g) | H₂O (ml) | HS-40 (ml) |
|---|---|---|---|---|
| 0.05 | 0.05 | 2 | 0.5 | 1.5 |

This material had all the attributes of the previous example in terms of hardening and creation of macropores in situ. Additionally, this composition had high strength in spite of the presence of large macropores. This cement set very well and rather quickly, producing great porosity and strength.

### Example 6

Another composition with enhanced strength was obtained by mixing PMP with magnesium phosphate in the ratio of 1:1 with HS-40 (colloidal silica suspended in water) as the liquid and pH buffered at 4.0. This composition is detailed in Table 4, below.

TABLE 4
PMP Composition with MgP and Colloidal Silica

| Mg powder | MgO | PMP | MgP | HS-40 | pH 4 buffer |
|---|---|---|---|---|---|
| (g) | (g) | (g) | (g) | (ml) | (ml) |
| 0.05 | 0.05 | 1 | 1 | 1.5 | 0.5 |

Acidic medium is important in certain situations. By mixing PMP and MgP in a 1:1 ratio, all the attributes of the previous compositions could be retained.

### Example 7

The composition from Example 6 was further modified by adding alginate to it. The alginate enhanced the handling strength as well as cell growth. This composition is detailed in Table 5, below.

TABLE 5
PMP Composition with Alginate

| Mg powder | MgO | PMP | HS-40 | pH 4 buffer | Alginate |
|---|---|---|---|---|---|
| (g) | (g) | (g) | (ml) | (ml) | (g) |
| 0.05 | 0.05 | 10 | 7.5 | 2.5 | 1 |

The sample solidified almost immediately, in a time period ranging from about 30 seconds to about 2 minutes. The composition was placed into DI H₂O after a few minutes and retained its structure. The resulting cement had great handling properties. The strength of cement was very high.

Certain embodiments of the methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A macroporous cement composition comprising a composition having pores made from a hydrogen-evolving process, wherein the pores range from about 100 μm to about 1 mm in size.

2. The macroporous cement composition of claim 1, wherein the hydrogen-evolving process is a reaction between a metal and water present in a liquid.

3. The macroporous cement composition of claim 2, wherein the metal is selected from the group consisting of magnesium, iron, and aluminum.

4. (canceled)

5. The macroporous cement composition of claim 3, wherein the magnesium is in the form of spherical particles.

6. The macroporous cement composition of claim 5, wherein the magnesium particles are about 45 μm in diameter.

7. The macroporous cement composition of claim 3, wherein the magnesium is present in an alloy with one or more of Al, Zn, or a rare-earth metal.

8. The macroporous cement composition of claim 1, wherein the liquid further comprises silica.

9. The macroporous cement composition of claim 1, wherein the macroporous cement composition further comprises a biopolymer or polysaccharide.

10. The macroporous cement composition of claim 9, wherein the macroporous cement composition comprises alginate.

11. The macroporous cement composition of claim 9, wherein the macroporous cement composition comprises chitosan.

12. The macroporous cement composition of claim 1, wherein the macroporous cement composition comprises a post-microwaved powder comprised of a calcium phosphate that has been irradiated by microwaves.

13. The macroporous cement composition of claim 1, wherein the macroporous cement composition comprises a calcium phosphate selected from the group consisting of monetite, brushite, hydroxyapatite, tetracalcium phosphate, tricalcium phosphate, and octacalcium phosphate.

14. The macroporous cement composition of claim 1, wherein the macroporous cement composition comprises a magnesium phosphate.

15. The macroporous cement composition of claim 1, wherein the macroporous cement composition comprises a strontium-doped calcium phosphate or a strontium-doped magnesium phosphate.

16. The macroporous cement composition of claim 1, wherein the macroporous cement composition is configured to be injectable during the hydrogen-evolving process.

17. The macroporous cement composition of claim 1, further comprising drug molecules in the pores.

18. The macroporous cement composition of claim 1, further comprising an antibiotic.

19. The macroporous cement composition of claim 18, wherein the antibiotic is selected from the group consisting of: clindamycin, tigecycline, vancomycin, ciprofloxacin, ofloxacin, sulfamethoxazole, trimethoprim/sulfamethoxazole, amoxicillin, penicillin V, penicillin G, procaine penicillin, benzathine penicillin, carbencillin, mezlocillin, ampicillin, piperacillin, bacampicillin, tiearcillin, ticarcillin, piperacillin/tazobactam, aztreonam, cefotetan, loracarbef, mefoxin, merrem, levofloxacin, lomefioxacin, primaxim, cycloserine, kanamycin, dicloxacillin, demeclocycline, minocycline, doxycycline, oxytetracycline, tobramycin, gentamicin, neomycin, amikacin, craramyein, nebcin, erythromycin/sulfisoxazole, netromycin, streptomycin, tobramycin, cefotaxime, cefuroxime, cefazoline, ceffibuten, ceffizoxime, cefaclor, cefopoerazone, cefprozil, cefadroxil monohydrate, ceftazidime, trimethoprim/sulfamethoxazole, cephalexin, cefazolin, cefamandole nafate, cefepime, cefonicid, sulfadiazine, norfloxacin, enoxacin, cefdinir, seromycin, ceftriaxone, cefixime, ceftazidime, clarithromycin, dirithromycin, methenamine, ethionamide, trovafioxacin, sparfloxacin, interfon-α, indinavir, ganciclovir, foscamet, lamivudine, famciclovir, rimantadine, zalcitabine, interferon-β, indinavir, ganciclovir, saquinavir, ritonavir, ribavirin, erythromycin, troleandomycin, azithromycin, eliiidamycin, colistin, amphotericin B, flucytosine, fluconazole, griseofulvin, grepafloxacin, ultramicrosize griseofulvin, terbinafine, ketoconazole, clotrimazole, dapsone, delavirdine, ziduvudine, amantadine, palivizumab, valacyclovir, didanosine, nelfinavir, nevirapine, ribavirin, cidofovir, pyrimethamine, metronidazole, furazolidone, atovaquone, stavudine, lamiduvine, acyclovir, mionazole, nystatin, itraconazole, chloroquine, pyrimethamine, mefloquine, hydroxychloroquine, capreomycin, permethrin, crotamiton, lindane, fluoro-uracil, ethambutol, rifabutin, isoniazid, aminosalicyclic acid, rifapentine, pyrazinamide, coenzoyl peroxide, chlorhexidine gluconate, sodium oxychlorosene, benzoyl peroxide, rifampin, rifampin/isoniazid, rifampin/isoniazid/pyrazinamide, nitrofurantoin, linezolid, nitrofurantoin, fosfomycin, nalidixic acid, atropine, oxytetracycline/sulfamethizole/phenazopyridine, mupirocin, chloramphenical, neomycin/polymyxin, tfimetorpim/polythyxin, tobramycin/dexamethasone, vidatabine, ciprofloxacin, ofioxacin, sulfacetamide, mupirocin, povidoneodine, gentamicin, nystatin, chloramphenicol, bacitracin, sulconazole, terbinafine, tetrachlorosalicylanilide, metronidazole, metromdazole, ciclopiroxolamine, clotrimazole, clotrimazole/betamethasone, butenafine, clotrimazole, nattifine, oxiconazole, selenium, econazole, penciclovir, and pharmaceutically acceptable salts thereof.

20. The macroporous cement composition of claim 1, further comprising a growth factor.

21. The macroporous cement composition of claim 20, wherein the growth factor is selected from the group consisting of BMP1, BMP2, BMP3, BMP4, BMP5, BMP6, BMP7, BMP8a, BMP8b, BMP10, and BMP15.

22. A cement composition comprising:
a powder component;
a liquid component; and
a porogen capable of reacting with the liquid component to produce a gas;
wherein the powder component is mixed with the porogen and the liquid component to form a gaseous solution, the gaseous solution setting to form a hardened porous mass.

23. A cement composition convertible by bodily processes into a macroporous cement composition of claim 1.

24. (canceled)

25. (canceled)

26. A method for making a cement composition comprising:
mixing a powder component with a liquid component and a metal to form a gaseous solution, wherein the metal is capable of reacting with water in a liquid to produce hydrogen gas; and
allowing the gaseous solution to set into a hardened porous mass, wherein the hydrogen gas bubbles create pores in the hardened mass.

27. A method for making a cement composition comprising:
mixing a powder component with a setting solution to form a paste;
irradiating the paste with microwaves to form post-microwaved powder;
mixing the post-microwaved powder with magnesium particles and a liquid to form hydrogen gas bubbles in a gaseous solution; and
allowing the gaseous solution to set into a hardened porous mass, wherein the hydrogen gas bubbles create pores in the hardened porous mass.

28. The method of claim 27, wherein the liquid comprises silica.

29. The method of claim 27, wherein at least one of alginate or chitosan is mixed with the post-microwaved powder, magnesium particles, and liquid.

30. The method of claim 27, wherein the hardening of the paste occurs simultaneously with the production of hydrogen bubbles in the gaseous solution.

31. The method of claim 27, wherein the composition is configured go be extruded through a syringe while pores are forming in the gaseous solution from the hydrogen bubbles.

32. (canceled)

33. A bone implant comprising the macroporous cement composition of claim 1.

34. A kit for making a macroporous cement composition comprising:
a first container housing a homogeneous powder comprising post-microwaved powder and magnesium; and
a second container housing an aqueous liquid.

35. The kit of claim 34, further comprising a syringe.
